(19) United States
(12) Patent Application Publication
(10) Pub. No.: US 2020/0311772 A1
(43) Pub. Date: Oct. 1, 2020

(54) TARGETED PATIENT ACQUISITION AND REPUTATION ENHANCEMENT

(71) Applicant: Social Climb LLC, Austin, TX (US)

(72) Inventors: Ty John Allen, Alpine, UT (US); Jared Bryan Maxwell, Herriman, UT (US); Michael John Franklin Walker, Lehi, UT (US)

(21) Appl. No.: 16/830,627

(22) Filed: Mar. 26, 2020

Related U.S. Application Data
(60) Provisional application No. 62/824,139, filed on Mar. 26, 2019.

(51) Int. Cl. G06Q 30/02; G16H 10/20; G06N 20/00

(52) U.S. Cl. CPC G06Q 30/0269; G06Q 30/0282; G06Q 30/0205; G06Q 30/0261; G06N 20/00; G06Q 30/0246; G06Q 30/0247; G16H 10/20; G06Q 30/0249; G06Q 30/0254

(57) ABSTRACT

This disclosure describes systems and methods for managing the reputation of a practitioner, such as a medical practitioner or another professional service provider, by requesting reviews after a procedure or other service has been provided. A targeted patient or client acquisition system leverages the strengthened reputation of the practitioner to acquire target patients. The targeted patient acquisition system identifies a target geographic region for targeted advertisement based on a weighted function of targeting factors for each of a plurality of geographic regions within a travel threshold of an office of the practitioner. A marketing management subsystem manages the electronic distribution of advertisements to individuals within the identified target geographic region. Calculated profitability metrics of new patients acquired via the targeted advertisements are used to adjust the target geographic region.

### RELATED APPLICATIONS

This application claims priority to U.S. Provisional Patent Application No. 62/824,139 filed on Mar. 26, 2019, titled “Professional Services Marketing System and Methods,” which is hereby incorporated by reference in its entirety.

### TECHNICAL FIELD

This disclosure relates to systems and methods for professional service advertising and practice management. Software-based systems may leverage cloud-based technologies, dedicated servers, and/or downloadable software to manage the advertising efforts of professional service providers.

### BRIEF DESCRIPTION OF THE DRAWINGS

This disclosure includes illustrative embodiments that are non-limiting and non-exhaustive. Reference is made to certain of such illustrative embodiments that are depicted in the figures described below.

FIG. 1 illustrates a simplified block diagram of interfaces between a patient acquisition system and external systems and entities, according to various embodiments.

FIG. 2 illustrates a basic block diagram of a patient acquisition system, according to one embodiment.

FIG. 3 illustrates a simplified flow diagram for targeted advertisement management for selective patient acquisition, according to various embodiments.

FIG. 4 illustrates an example graphical user interface (GUI) to facilitate the geographic visualization of the quality assessments of existing and/or prior patients, according to various embodiments.

FIG. 5 illustrates an example GUI for summarized visualization of potential patient information associated with a target geographic region, according to various embodiments.

FIG. 6A illustrates a block diagram of an example of a system for targeted patient acquisition and reputation enhancement, according to various embodiments.

FIG. 6B illustrates a block diagram of another example embodiment of a system for targeted patient acquisition and reputation enhancement, according to various embodiments.

FIG. 7 illustrates a flow chart of a method for geographically targeted advertisement and reputation enhancement, according to various embodiments.

### DETAILED DESCRIPTION

Marketing a physician and/or medical practice to potential patients can require considerable effort. Historically, physicians have spent most of their marketing efforts on other medical providers, payers, and/or medical networks to encourage such entities to provide new patients on a referral basis. With the advent of social media sites, Internet search engines, consumer reporting tools, and the like, the patient acquisition effort has increasingly turned to direct marketing to self-referring potential patients.

Large medical practices (such hospitals and networks of doctors) may have dedicated marketing personnel to manage the online reputation of the practice as a whole and the individual practitioners associated therewith. The marketing personnel may also manage advertisement efforts, including marketing efforts to other medical practices, payers, other medical networks, and direct advertisements to self-referring potential patients. Small medical practices and solo practitioners may not have dedicated marketing personnel and instead rely on external advertising agencies or basic online advertising tools.

The presently described systems and methods can be used by large medical practices via integration into existing processes, customer relationship management (CRM) systems, and/or other existing electronic systems. Smaller practices and individual practitioners may also use the presently described systems and methods for reputation enhancement and targeted patient acquisition. Smaller practices and solo practitioners may especially benefit from the ability to attract high-quality patients and manage their online reputation without expending a significant amount of time or hiring dedicated personnel.

Many of the embodiments described herein refer to improving the reputation of a practitioner and acquiring new patients for the practitioner. However, it is appreciated that many of the embodiments described herein can be applied to individual practitioners or to a practice as a whole. Accordingly, while the specific examples used herein generally refer to a “practitioner,” the term is used to encompass individual practitioners or a practice of many practitioners. Similarly, many of the examples described herein are provided in the context of healthcare practitioners and healthcare practices. However, it is appreciated that the systems and methods described herein may be applied to a wide range of professional services and practices. For instance, the term “practitioner” is understood to encompass a wide variety of professional service provides including, without limitation, physicians, surgeons, therapists, architects, accountants, engineers, doctors, lawyers, tax professionals, appraisers, investment managers, IT consultants, veterinary physicians, and the like.

Similarly, while the term “patient” makes sense in the context of healthcare practitioners and is used herein in many examples, the term is used in a broader sense to encompass any individual or entity requesting the professional services of any of the above-described professional service provides.

Various embodiments of the systems and methods described herein facilitate automatic and/or semi-automatic organic improvement in the reputation of a practitioner, the organic increase in the online visibility of a practitioner, patient quality analysis, potential patient analysis, marketing spend management, return on advertising spend (ROAS) analysis, paid-for targeted increases in the visibility to target patients, and targeted advertisement to potential patients.

Various systems and methods are described herein for managing the reputation of a practitioner (or another professional service provider). An automated system may request that patients provide a review or other feedback after a procedure has been performed or another service has been provided. Positive reviews may be posted on websites to improve the overall reputation of the practitioner and/or enhance the organic visibility of the practitioner. Geographically targeted activities may be specifically utilized to enhance the reputation and/or increase the visibility of the practitioner within a target geographic region or target geographic regions. In some embodiments, negative reviews may be handled directly and not posted to publicly visible websites.

A targeted patient acquisition system may leverage the strengthened reputation of the practitioner to acquire target patients. The targeted patient acquisition system may identify a target geographic region for targeted advertisement based on, for example, a weighted function of targeting factors for each of a plurality of geographic regions within a travel threshold of an office of the practitioner. A marketing management subsystem may manage the electronic distribution of advertisements to individuals within the identified target geographic region. The system may utilize the calculated profitability metrics of new patients acquired via the targeted advertisements to adjust the target geographic region.

Thus, according to various embodiments, a patient acquisition system may include a practitioner reputation management subsystem, a target patient identification subsystem, an advertisement management subsystem, a tracking subsystem, a profitability return on advertising spend (PROAS) calculation subsystem, a reporting subsystem, and/or a practitioner interface subsystem. Each of the above-listed subsystems, and the other subsystems and modules described herein, may be embodied as stand-alone subsystems, combined, or divided into sub-subsystems.

According to various embodiments, a practitioner reputation management subsystem may automatically detect that a patient has received services from a particular practitioner and request that the patient provide a review that can be posted on a publicly visible website. The practitioner reputation management subsystem may, for example, direct existing patients to provide reviews on one or more publicly visible websites. Some such websites may specifically curate reviews of service providers. In some embodiments, the practitioner reputation management subsystem may direct some patients to post reviews on a first website and other patients to post reviews on a second website.

In various embodiments, a target patient identification subsystem may identify a target geographic region for targeted advertisement. The target geographic region may be selected based on a weighted function of any combination or permutation of a wide variety of targeting factors. The geographic regions evaluated or eligible for selection as the target geographic region may be limited to those that are within a travel threshold of a medical facility of the practitioner (or another office of a different type of professional service provider). Examples of weighted targeting factors for each of the eligible geographic regions include, for example, profitability metrics of prior patients, average household income metrics, an estimated total market share metric of a type of procedure offered by the practitioner, an estimated percentage metric of the total market share already captured by the practitioner, and/or demographic metrics such as age, gender, marital status, number of children, race, occupation, education level, and/or the like. In various embodiments, the system may utilize a national data set of information for individuals and households to identify individuals having the target factors.

It is appreciated that the target geographic region is not necessarily a contiguous geographic region on a map. Rather, the target geographic region may comprise any number of discontiguous geographic regions as selected by the target patient identification subsystem. For example, the target geographic region may include several neighborhoods or zip codes within a travel distance threshold that satisfy the targeting factor analysis.

In various embodiments, an advertisement management subsystem may authorize the electronic distribution of an advertisement within the target geographic region. In various embodiments, the advertisement management subsystem may receive an advertisement, evaluate a remaining advertising budget of the practitioner, determine availability in the schedule of the practitioner, and verify that no conflicting or competitive advertisements are currently being distributed by the system. After verification, the advertisement management subsystem may authorize the advertisement for electronic distribution (e.g., push, post, or otherwise cause the advertisement to be distributed).

The system may also include a tracking subsystem to identify a conversion rate of viewers of the advertisement that become new patients. The system may also calculate a profitability metric, such as a profitability return on advertising spend (PROAS) metric. Many online advertisements are evaluated based on conversion rates and click-through-rates. For product sales, conversion rates equate to profits since each product sold can be evaluated in terms of fixed revenue and profit. Similarly, for large professional service providers, such as hospitals or doctor networks, the large number of patients treated each year allows them to develop average revenue metrics and average profitability metrics for each patient. Accordingly, traditional conversion rate analysis of online advertising may be sufficient.

In contrast, small practices and solo practitioners are only able to treat a relatively small number of patients each year. The profitability (or lack thereof) of each patient has a relatively large impact on the overall success of the practitioner or practice. Accordingly, the presently described systems and methods evaluate the profits associated with the patients acquired through the targeted advertisements—and not merely the fact that the individuals became patients. For example, a practitioner may find that patients of a certain age and with a particular type of insurance are highly profitable and result in few complications. In contrast, other patients having common characteristics may pay less for procedures, have inferior insurance, and/or encounter more frequent complications that can be time-consuming or expensive for the practitioner.

Accordingly, the system may also include a profitability analysis subsystem to evaluate the profitability of the patients acquired via the targeted advertisements and adjust the weightings applied to the targeting factors used to select the target geographic region. For example, the profitability subsystem may utilize a machine learning model trained using associations between the weightings applied to the targeting factors and the profitability metric calculated by a PROAS calculation subsystem to adjust the weightings applied to the targeting factors.

In various embodiments, a reporting subsystem may generate a graphical user interface (GUI) to illustrate the profitability and residence location of current patients overlaid on a map. The reporting system may additionally, or alternatively, illustrate metrics associated with the targeting factors, the various geographic regions considered for eligibility as the target geographic region, and a selected target geographic region. The system may provide a practitioner interface subsystem that allows the practitioner to adjust a marketing budget and/or visualize the calculated profitability metrics associated with the new patients acquired via the advertisement in the target geographic region. As described herein, the system may interface with existing practice management systems to facilitate scheduling, patient identification, data mining and collection, contact information retrieval, and/or other information contained therein.

In some embodiments, a GUI may provide an overlay of actual (e.g., existing) patient traffic on predictive models. For example, the GUI may generate a map and overlay the social media presence, listings, reputation rankings, reviews, and other online activity associated with a practitioner and/or practice. The overlay illustrates the “coverage” a practitioner or practice has established within various geographic regions. The system may overlay past and current online activity for any period of time to provide a graphical display that illustrates the improved reputational value and coverage provided by the system. The GUI may display the percentage of the recipients of the advertisements that became new patients and the profitability associated therewith. The GUI may also include conversion rates of organic listings based on the enhanced reputation provided by the system as compared to the paid-for advertisements (including GMB placements).

Some of the infrastructure that can be used with embodiments disclosed herein is already available, such as general-purpose computers, computer programming tools and techniques, digital storage media, and communications networks. A computer may include a processor, such as a microprocessor, microcontroller, logic circuitry, or the like. The processor may include a special purpose processing device, such as an ASIC, a PAL, a PLA, a PLD, an FPGA, or another customized or programmable device. The computer may also include a computer-readable storage device, such as non-volatile memory, static RAM, dynamic RAM, ROM, CD-ROM, disk, tape, magnetic, optical, flash memory, or another (non-transitory) computer-readable storage medium.

Aspects of certain embodiments described herein may be implemented as software modules or components. As used herein, a software module or component may include any type of computer instruction or computer-executable code located within or on a computer-readable storage medium. A software module may, for instance, comprise one or more physical or logical blocks of computer instructions, which may be organized as a routine, program, object, component, data structure, etc. that perform one or more tasks or implement particular data types.

A particular software module may comprise disparate instructions stored in different locations of a computer-readable storage medium, which together implement the described functionality of the module. Indeed, a module may comprise a single instruction or many instructions, and may be distributed over several different code segments, among different programs, and across several computer-readable storage media. Some embodiments may be practiced in a distributed computing environment where tasks are performed by a remote processing device linked through a communications network. In a distributed computing environment, software modules may be located in local and/or remote computer-readable storage media. In addition, data being tied or rendered together in a database record reside in the same computer-readable storage medium, or across several computer-readable storage media, and may be linked together in fields of a record in a database across a network.

Some of the embodiments of the disclosure can be understood by reference to the drawings, but such drawings are merely examples and should not be construed as limiting the subject matter described or claimed herein. The components of the disclosed embodiments, as generally described and illustrated in the figures herein, could be arranged and designed in a wide variety of different configurations. Thus, the detailed description of the embodiments of the systems and methods of the disclosure is not intended to limit the scope of the disclosure, as claimed, but is merely representative of possible embodiments. Well-known structures, materials, or operations are not shown or described in detail to avoid obscuring aspects of this disclosure. In addition, the elements of described and illustrated methods do not necessarily need to be executed in any specific order, or even sequentially, nor need such element be executed only once unless otherwise specified.

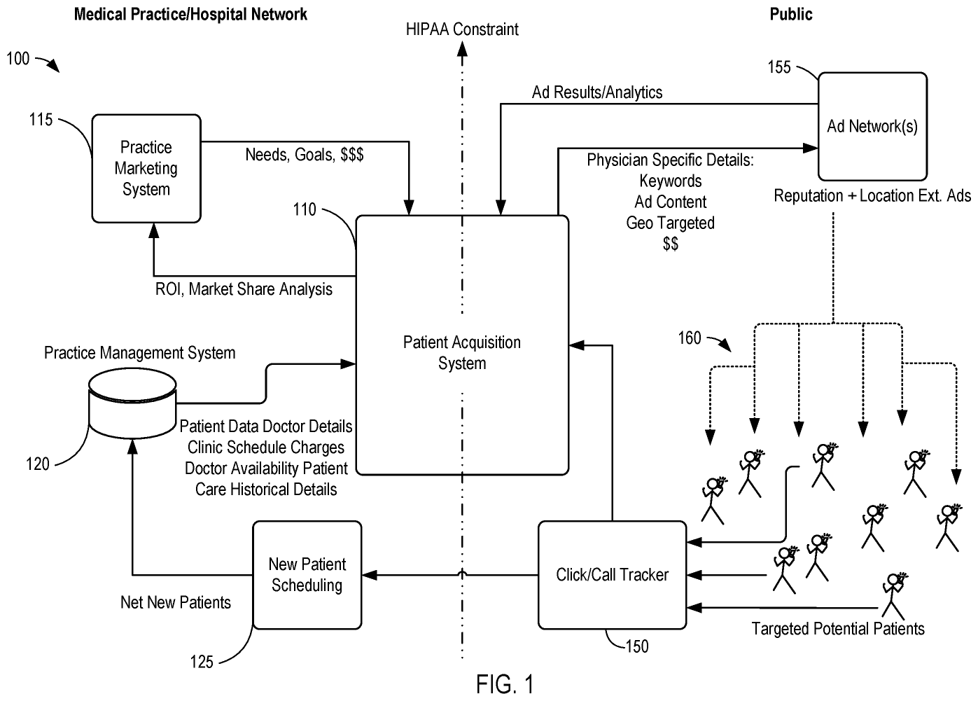

FIG. 1 illustrates a simplified block diagram 100 of interfaces between a patient acquisition system 110 and external systems and entities, according to various embodiments. As illustrated, the patient acquisition system 110 may interface with a practice marketing system 115 to receive an advertisement for electronic distribution in a target market. In some embodiments, the patient acquisition system 110 may include a graphical user interface (GUI) through which human managers or the practitioners themselves may specify the meets and bounds of the advertising campaign.

The patient acquisition system 110 may include a patient data analysis module to analyze patient data and information pertaining to prior, current and/or potential patients. The patient acquisition system 110 may determine the return on investment of current patients, market share in a particular location, location information of current and/or characteristics of past patients identified as “target” or “high-quality” patient (e.g., patients deemed “desirable” or “best”), household income of potential patients, number of individuals in the homes of potential patients, and/or other information. The patient acquisition system 110 may establish or receive information identifying the goals for new patient acquisition and/or financial goals.

In various embodiments, the patient acquisition system 110 may maintain, access, or update patient data, historical patient care data, physician information, billing information, physician availability, and/or the like. For example, in the illustrated embodiment, the patient acquisition system 110 is in communication with a practice management system 120 of the practitioner.

The patient acquisition system 110 may access the practice management system 120 or receive a signal from the practice management system 120 to determine when new patients are needed (e.g., when the practitioner's schedule has an upcoming opening). A target patient identification subsystem of the patient acquisition system 110 may determine a target geographic region within which potential patients should receive targeted advertisements. The patient acquisition system 110 may transmit the advertisement content, keywords, the target geographic region, target spend, and/or other information to advertising network systems 155.

The advertisements may be pushed for electronic distribution through any of a wide variety of formats, including through social media, Internet search engines, and/or consumer report engines. Individual potential patients 160 within the target geographic regions may view the advertisement and contact the practitioner (or the practitioner's office). A new patient scheduling module 125 may facilitate scheduling of new patients through the practice management system 120. The patient acquisition system 110 may include or be in communication with a tracking subsystem 150 and/or profitability return on advertising spend subsystems to calculate a return on investment (ROI), return on advertising spend (ROAS), and/or a profitability return on advertising spend (PROAS).

Accordingly, the patient acquisition system 110 may collect or determine information regarding the success rate of advertisements transmitted to different target geographic regions for different practices, practitioners, and at different times. The patient acquisition system 110 may utilize this information, in conjunction with the information in the practice management system, to adjust the target geographical region, advertising budget, and the like. For example, the patient acquisition system 110 may utilize patient data, doctor details, clinic schedules, charges, accounts receivable, payments, adjustments to payments, write-offs, write-downs, patient care details, and the like. The patient acquisition system 110 may cause advertisements to be displayed in geographically targeted locations and evaluate the efficacy thereof via a continuously learning system to deliver an iteratively optimized flow of target patients to fill openings in the schedules of practitioners. The advertisements may be, for example, location extension ads, such as those provided by Google™ and/or those offered in connection with Google My Business (GMB) pages.

The patient acquisition system 110 may collect data regarding potential patients who respond to advertisements. For potential patients who elect to become patients, the patient acquisition system 110 may communicate with the practice management system 120 to set an appointment at a date and time when the practitioner is available. Patient information and attendant scheduling information may also be communicated to the patient acquisition system 110. The patient acquisition system 110 uses potential patient response information to improve future advertisement keywords, content, and location-based postings. Artificial intelligence, machine learning, and/or other intelligent systems and algorithms may learn from interactions and received information to improve performance and/or increase the accuracy with which target clients are curated.

According to various embodiments, the patient acquisition system 110 may interact with the practice marketing system 115, practice management system 120, advertising network systems 155, and click/call tracking services via application programming interfaces (APIs). For example, the patient acquisition system 110 may interface via APIs with the advertising platforms offered by any of a wide variety of companies, including Google™, Facebook™, Microsoft™, and the like.

To provide a specific example, each instance of an advertisement (e.g., web advertisement, google extension add, GMB page, etc. displayed to a potential patient may be associated with a unique phone number, uniform resource locator (URL), and/or Urchin Tracking Module (UTM) identifier as part of Google Analytics or an equivalent web advertisement placement and tracking service. Using Google Analytics as an example, unique UTM ID (and/or phone number) enables the system to track each potential patient that receives an advertisement, click-through rates, and conversion rates.

A marketing journey may be created and monitored for each potential patient using data available via a web advertisement system, such as google analytics, and the integrated practice management system of the medical practitioner or other professional service entity. The marketing journey may detail and track displayed advertisements, click rates, time spent viewing webpages, phone calls made, appointments made, care or other services provided, payments made (or not made), etc. Artificial intelligence and machine learning algorithms may further refine each step of the marketing journey to curate advertisements and refine the target geographic region to attract a set of clients that have desirable characteristics/traits and/or the ability to pay for the services provided, all while minimizing ad-spend through selective and targeted advertisement placement and tracking.

For instance, when a potential patient clicks on an advertisement or a GMB listing and chooses to call the phone number associated with a particular physician, the potential patient's phone number and the call details may be tracked and recorded. The patient acquisition system 110 may later search the practice management system 120 to match the tracked and recorded phone numbers associated with advertising efforts with those phone numbers associated with actual patients. The patient acquisition system 110 may determine a profitability associated with each patient to refine the set of characteristics used to define the “best” or target patients. The patient acquisition system 110 may use this information to report the effectiveness of the advertising dollars, advertisement placement locations, timing, keywords, etc.

As another example, a patient dialing in, initiating a chat, or otherwise communicating to set up an appointment, obtain more information, and/or otherwise procure or learn more about the services may be seamlessly connected with the medical provider's office. For example, a patient dialing the unique phone number that is part of an advertisement may be forwarded to the phone number of the doctor's office without interruption. The potential patient may not even be aware of the forwarding. The length of the call, who answered the call, the results of the call, and other details related to the call may be tracked, recorded, and associated with the unique details of the conversion.

Over time, the system may correlate actual patient phone numbers with those numbers identified as calling the medical facility based on a particular advertising campaign. The system may evaluate the click-through rates, conversion rates, ability to attract target patients, ability to avoid undesirable patients, cost to obtain new clients, and the like may be evaluated to quantify the value of the advertising campaign and GMB pages in terms of the patient names, the dollars collected, the procedures performed, the age of patients, the gender of patients, the timeliness of patient payments, the insurance payouts, the insurance adjustments, etc.

Patients that choose to click on a URL, initiate an online chat, schedule a consultation, send an email or other message through an online portal, etc. may be tracked in a similar way. A unique ID for each advertisement and/or GMB page may be recorded in the practice's web analytics system and compared with the results available within the practice management system. By using data available from the connected practice management system, the advertisement placement and analysis tools are able to learn quickly and identify the most successful advertising campaigns and approaches that result in measurable successes in terms of procedures, patients, and payments.

The system may provide real, measurable results for tracking the value of each advertisement, each advertising campaign, each GMB page, etc. The names, addresses, current procedural terminology (CPT) codes, procedure descriptions, revenue generated, profits made, etc. can be correlated with specific advertisements, advertising campaigns, GMB pages, keywords, demographics, etc. The system may periodically, on-demand, or continually provide a marketing report card that is dynamically updated to show the value of individual advertisements or advertising campaigns. A return on investment for all spend on reputation and/or targeted advertisements may be calculated and reported in the marketing report.

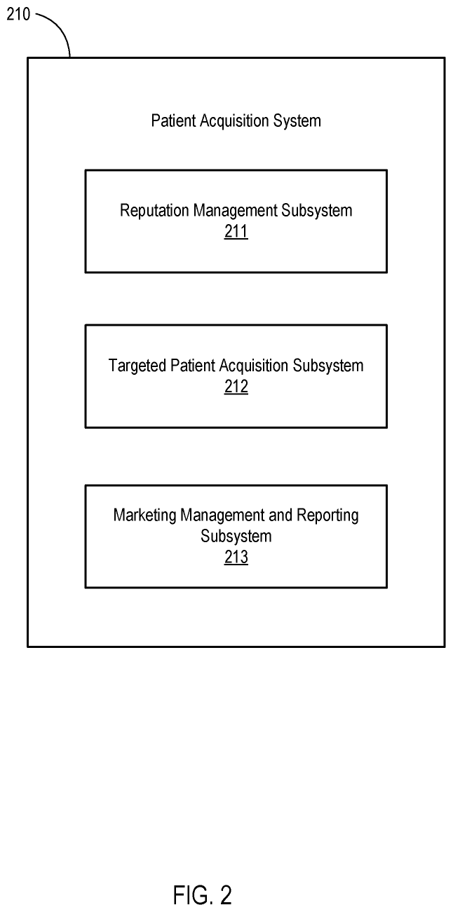

FIG. 2 illustrates a basic block diagram of a patient acquisition system 210, according to one embodiment. As illustrated, the patient acquisition system 210 may include a reputation management subsystem 211 that automatically detects that a patient has received services from a particular practitioner and request that the patient provide a review that can be posted on a publicly visible website. The practitioner reputation management subsystem 211 may, for example, direct existing patients to provide reviews on one or more publicly visible websites and other platforms. In some embodiments, the practitioner reputation management subsystem 211 may direct different patients to different websites to selectively enhance the reputation of the practitioner across a variety of websites and platforms.

Improving the organic reputation of the practitioner increases the organic visibility of the practitioner when potential clients search for the services offered by the practitioner. In some embodiments, the system may increase the visibility of the practitioner by paying for search result placement when a potential patient that lives within the target geographical area searches for services offered by the practitioner.

The patient acquisition system 210 may also include a targeted patient acquisition subsystem 212 to identify a target geographic region for targeted advertisement. As previously described, the target geographic region may be selected based on a weighted function of any combination or permutation of a wide variety of targeting factors, such as information collected from the practice marketing system 115 (FIG. 1) and/or practice management system 120 (FIG. 1).

Depending on the specific services, treatments, and procedures offered by the practitioner, the target geographic region may be limited to distances that are within a travel threshold (e.g., time or miles) of a medical facility of the practitioner. As previously described, the target geographic region may comprise a plurality of discontiguous geographic regions, neighborhoods, zip codes, communities, cities, counties, or the like.

The patient acquisition system 210 may also include a marketing management and reporting subsystem 213 to interface with marketing individuals, marking systems, and/or the practitioners themselves. The marketing management and reporting subsystem 213 may generate any of the various GUIs and/or reports described herein. For instance, marketing management and reporting subsystem 213 may report profitability metrics associated with the new patients acquired via advertisements in the target geographic region. The marketing management and reporting subsystem 213 may also notify managers, administrators, marketing personnel, and/or the practitioners themselves of growth opportunities available if the advertising budget is increased.

Accordingly, the system can provide a graphical illustration and/or numerical data demonstrating the past return on investment and identifying specific marketing plans for increasing reputation and/or targeted patient acquisition. For example, the system may identify growth opportunities within specific geographic areas identified to have a high likelihood of providing target new patients. A growth opportunity report may be provided to users of the system that includes one or more advertising campaign for a targeted geographic region.

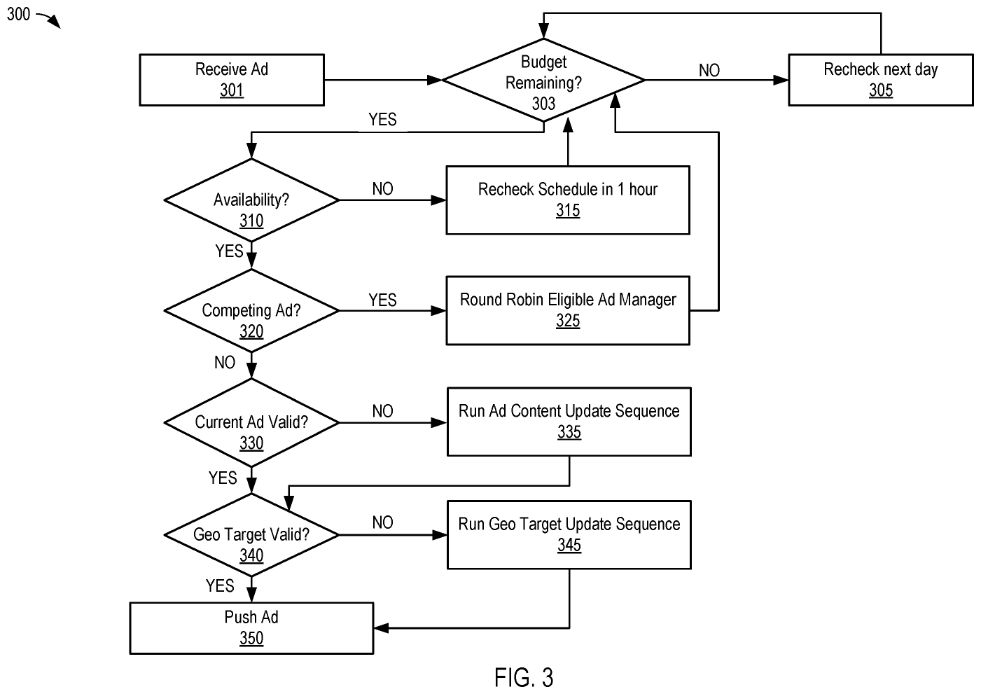

FIG. 3 illustrates a simplified flow diagram 300 for targeted advertisement management for selective patient acquisition, according to various embodiments. An advertisement may be received, at 301, and the system may determine if the advertising campaign has budget remaining, at 303. If there is no budget remaining for the current time period (e.g., day), then the system may recheck the next day, at 305, to see if there is budget remaining, at 303. With budget remaining, at 303, the system may determine if there is availability, at 310, in a calendaring system of the practitioner (e.g., by checking with the practice management system 120 (FIG. 1).

In various examples, the system may determine that the practitioner has an opening in their schedule, at 310, for a new patient at a configurable time in the future. The configured length of time in the future may be selected to ensure advertisement efforts have enough lead-time to provide a high likelihood of scheduling a target client. If an opening in the schedule is not available, the system waits, at 315, a configurable amount of time before reattempting to advertise. While an opening in the schedule is not available, advertisements are not placed. This reduces the likelihood of having would-be patients frustrated by an inability to schedule an appointment in the near future and also reduces the amount of money wasted on advertising when new patients are not needed and/or can't be serviced.

In the illustrated embodiment, if an opening in the physician's schedule exists, at 310, the system verifies, at 320, that an advertisement would not compete with an advertisement from a physician within the same medical practice (or, more generically, a conflicting advertisement for a related professional service provider). If the advertisement would compete, at 325, with an advertisement from the same medical practice, a new physician is selected, and the module reattempts to advertise (e.g., execute a round-robin eligible advertisement manager). If the proposed advertisement does not compete, at 320, with others in the same medical practice, the advertisement content is evaluated, at 330. If the advertisement content is deemed inappropriate or invalid, at 330, the advertisement content is updated, at 335.

With appropriate or valid advertisement content, the system determines whether the geographical region associated with the advertisement content is valid, at 340. The geographical region associated with the advertisement content may be updated, at 345, if needed, and then the advertisement content may be pushed, at 350, for digital publication. Accordingly, given a sufficient budget, practitioner availability, no advertisement conflicts, valid content, and valid geographical region information, the system sends the advertisement to appropriate media channels.

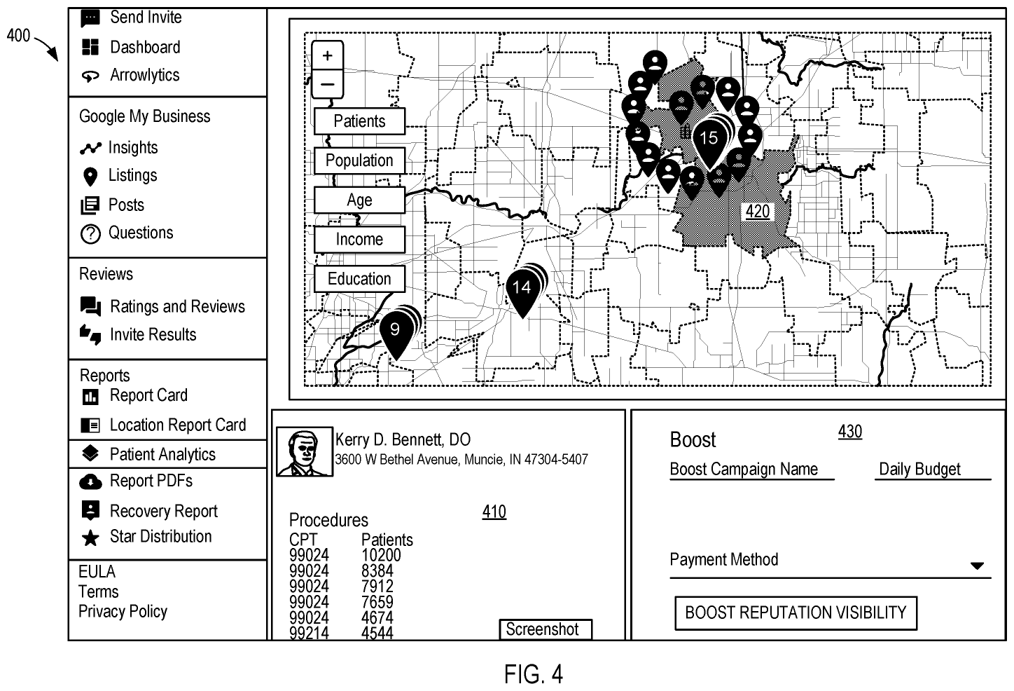

FIG. 4 illustrates an example graphical user interface (GUI) 400 to facilitate the geographic visualization of the quality assessments of existing and/or prior patients, according to various embodiments. In one embodiment, the patient acquisition system may use the displayed information to identify and/or select potential patients with desirable attributes (i.e., target patients). The system may then target these potential patients to receive individualized and/or personalized advertisements in an effort to acquire them as patients. Data regarding these patients may be collected, purchased, and/or acquired using any of a wide variety of data sources.

In the illustrated embodiment, practitioner specific information may be displayed in a practitioner windows 410. Additionally, a map window may display a graphic of the locations of patients on a map and quality metrics associated with the identified patients or potential patients. A target geographic region 420 may be identified for the distribution of advertisements. A boost window 430 may display an existing advertising campaign and/or allow for a user to create an advertising campaign for the target geographic region. The target geographic region 420 may, in some embodiments, include discontiguous regions number 9 and 14 as well.

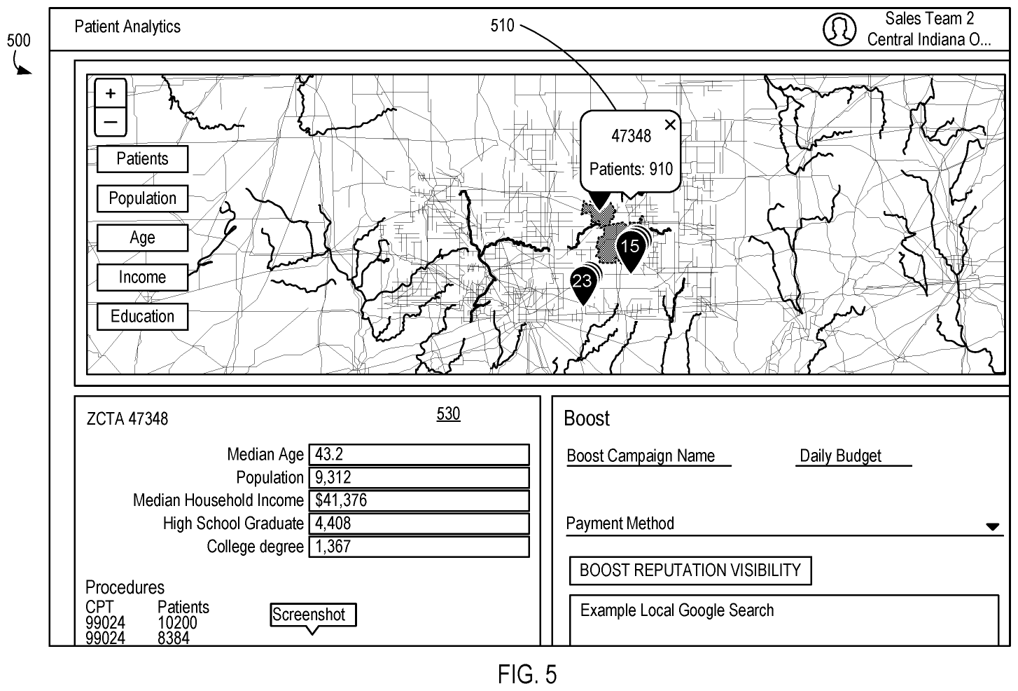

FIG. 5 illustrates an example GUI 500 for summarized visualization of potential patient information 530 associated with a target geographic region 510, according to various embodiments. In various embodiments, the system may use this information to identify and/or select potential patients with desirable attributes, making them “target patients.” The system may then target advertisements to these potential patients. In some embodiments, the system may receive individualized and/or personalized advertisements in an effort to acquire them as patients.

In various embodiments, advertisements may be tailored to leverage the practitioner's reputation. For example, the advertisement may include a reference to reputation, include a graphical indication, numerical representation, textual review, and/or recorded testimonial. A reputation may be location-based to provide information regarding how others in the same or nearby locations “feel” about the practitioner and/or associated practice. Furthermore, the system may maximize the return on advertising spend by waiting to deploy advertisements until an organic reputation of the practitioner has been established. Accordingly, the system may intentionally delay pushing the advertisements to the target geographic region until a threshold number of reviews and/or a threshold rating level are established for the practitioner.

In some embodiments, the system includes a reputation management module or subsystem to proactively increase the reputation of a practitioner and/or associated practice. For example, a reputation management module or subsystem may generate social media posts, monitor content, respond to comments, request feedback, collect survey information, collect and distribute patient reviews, etc. In some embodiments, location information may comprise GPS coordinates, addresses, area codes, voter districts, and/or zip codes. In some embodiments, the locations may be based on arbitrary zones dedicated to a particular medical practitioner or medical practice.

In some embodiments, the system uses artificial intelligence and/or machine learning to improve efficiency and/or the accuracy of identifying target clients. The success of advertisement content, location target, keywords, length of time before physician availability, and/or other advertisement characteristics are measured to determine advertising parameters that result in new patients and what parameters did not. Characteristics and parameters that result in new patients with acceptable profitably metrics may continue to be used and/or have increased weightings in a weighted function analysis. Those characteristics and parameters that are determined not to be correlated with targeted patient acquisition may be rejected for future use and/or weightings used in a weighted function analysis may be decreased.

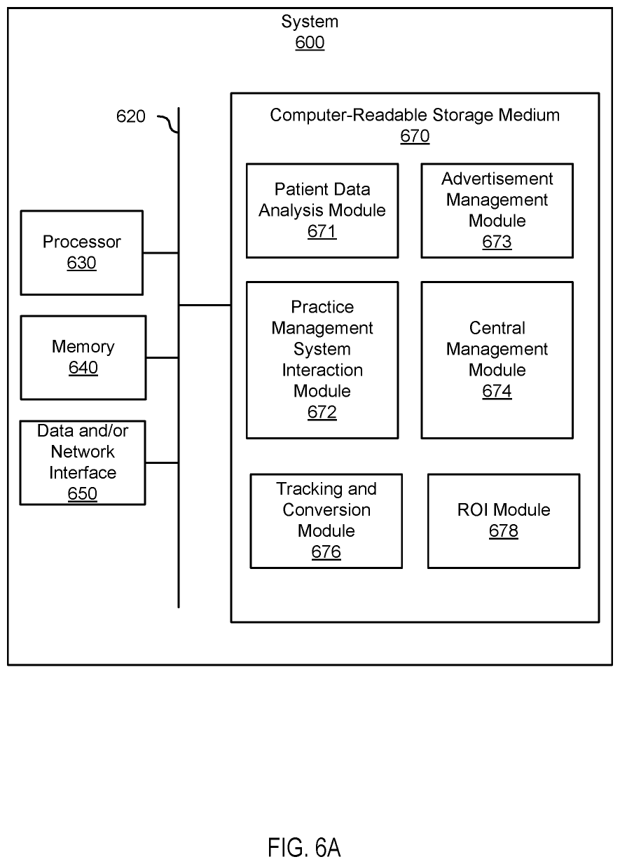

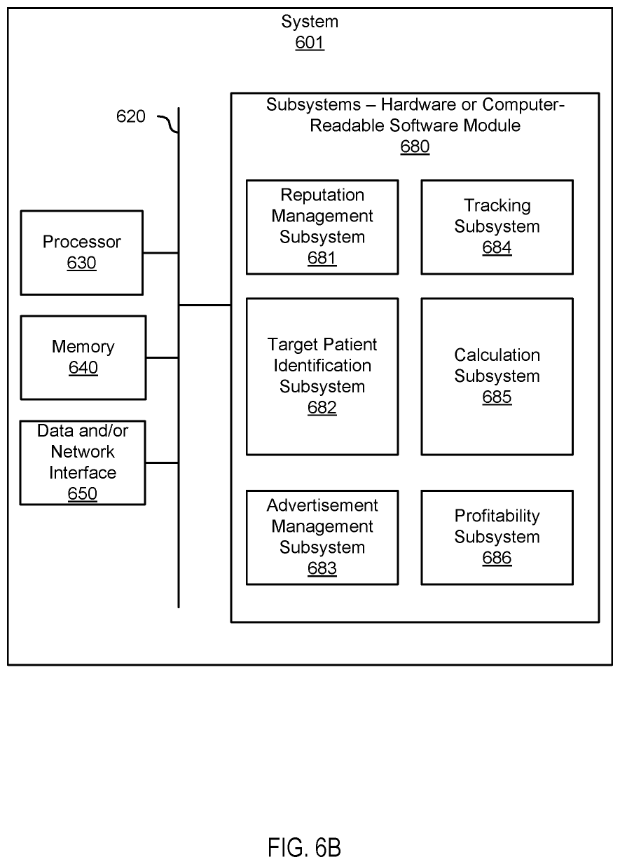

FIG. 6A illustrates a computer system 600 that includes a processor 630, a memory 640, a data and/or network interface 650, and a computer-readable storage medium 670 connected via a bus 620. The computer-readable storage medium 670 may include various modules illustrated as software modules that may alternatively be referred to as subsystems. In some embodiments, portions of or complete modules and subsystems described herein may be implemented in software, firmware, hardware, or combinations thereof.

A patient data analysis module 671 may receive, collect, and/or analyze patient data. A practice management system interaction module 672 may be configured to interact with a medical provider's existing practice management system or at least portion thereof, such as a calendar and scheduling portion. An advertisement management module 673 may manage the placement, timing, and content of advertisements. A central management module 674 may coordinate advertising efforts and use artificial intelligence and/or machine learning algorithms to increase the efficacy of the advertising efforts to cost-effectively curate target clients and schedule them during holes in a provider's existing schedule.

A tracking and conversion module 676 may track individual advisement successes and failures. For example, the tracking and conversion module 676 may track unique phone numbers, URLs, and/or UTM IDs of advertisements and GMB pages. The tracking and conversion module 676 may work with the practice management system interaction module 672 (optionally through the central management module 674) to seamlessly connect potential patients and the professional service provider and track the success of individual advertisements and advertising campaigns.

A return on investment (“ROI”) module 678 may evaluate the effectiveness of the advertisement by comparing tracked advertisements and advertising campaigns with patient data available in the practice management system interaction module 672. The ROI module 678 may report the success and failure of advertising campaigns and provide useful reporting data to the customer (to see results) and/or to training systems intended to learn from the successes and failures and adapt (e.g., through machine learning) the advertising campaign to increase the ROI. The ROI module 678 may provide quantifiable data tracking the value of each advertisement and GMB page. The names, addresses, CPT codes/procedures, revenue generated, and other metrics may be collected and tracked for each advertisement (including GMB pages) that result in a new patient.

FIG. 6B illustrates a block diagram of another example embodiment of a system 601 for targeted patient acquisition and reputation enhancement, according to various embodiments. As illustrated, the computer system 601 includes a processor 630, a memory 640, a data and/or network interface 650, and subsystems 680 connected via a bus 620.

The subsystems 680 include a reputation management subsystem 681 to request and manage the procurement of reviews from patients that receive services from a practitioner. A target patient identification subsystem 682 may facilitate the identification of a target geographic region. For example, the target patient identification subsystem 682 may implement a weighted function analysis of each of a plurality of geographic regions that are located within a travel threshold of a medical facility of the practitioner. The target patient identification subsystem 682 may implement a weighted function analysis of any number of targeting factors, including various demographic metrics.

An advertisement management subsystem 683 may manage the electronic distribution of an advertisement within the target geographic region. In some embodiments, the advertisement management subsystem 683 may implement retargeting automation of advertisements on platforms like the Facebook™ and Instagram™ platforms. Tracking subsystem 684 may identify a conversion rate of viewers of the advertisement that become new patients. A calculation subsystem 685 may calculate a profitability metric of the new patients acquired via the advertisement in the target geographic region. A profitability subsystem 686 may adjust weightings applied to the targeting factors based on the calculated profitability metric. The system may further include a reporting subsystem to generate a marketing report card that illustrates the value, including profitability measured by actual reimbursements for procedures performed, for the marketing activities (advertisement spend).

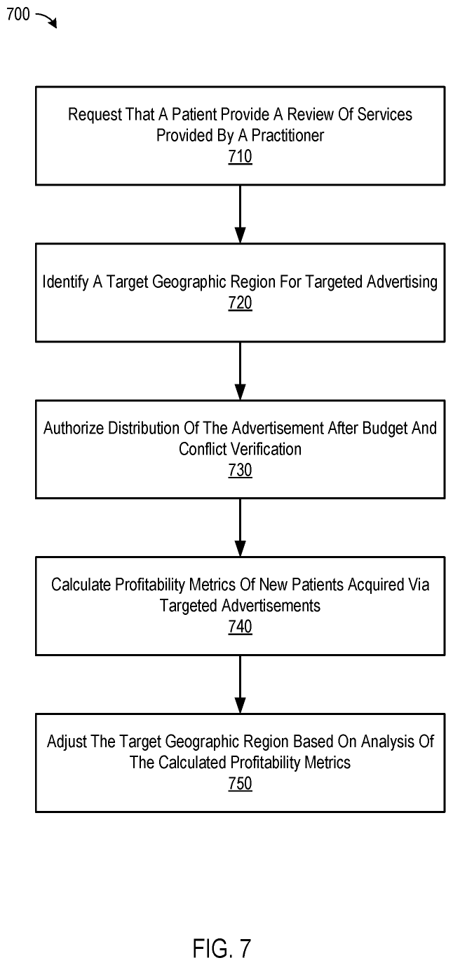

FIG. 7 illustrates a flow chart of a method 700 for geographically targeted advertisement and reputation enhancement, according to various embodiments. The elements of the method 700 may be implemented by a system having a combination of hardware, firmware, and/or processor-executable instructions. The system may request, at 710, that patients provide a review of services provided by a practitioner. The system may identify, at 720, a target geographic region for targeted advertising. The system may authorize, at 730, or otherwise cause the advertisement to be disseminated to the target geographic region. The system may calculate, at 740, profitability metrics of new patients acquired via targeted advertisements. The system may adjust, at 750, the target geographic region based on the analysis of the calculated profitability metrics.

Specific examples of the disclosure are described above and illustrated in the figures. It is, however, appreciated that many adaptations and modifications could be made to the specific configurations and components detailed above. In some cases, well-known features, structures, and/or operations are not shown or described in detail. Furthermore, the described features, structures, or operations may be combined in any suitable manner. It is also appreciated that the components of the examples, as generally described, and as described in conjunction with the figures herein, can be arranged and designed in a wide variety of different configurations. Thus, all feasible permutations and combinations of examples are contemplated. Furthermore, it is appreciated that changes may be made to the details of the above-described examples without departing from the underlying principles thereof.

In the description above, various features are sometimes grouped together in a single example, figure, or description thereof for the purpose of streamlining the disclosure. This method of disclosure, however, is not to be interpreted as reflecting an intention that any claim now presented or presented in the future requires more features than those expressly recited in that claim. Rather, it is appreciated that inventive aspects lie in a combination of fewer than all features of any single foregoing disclosed example. The claims are hereby expressly incorporated into this Detailed Description, with each claim standing on its own as a separate example. This disclosure includes all permutations and combinations of the independent claims with their dependent claims.

What is claimed is:

1. A patient acquisition system, comprising:
a practitioner reputation management subsystem to automatically request that existing patients, subsequent to a procedure performed by the practitioner, post a review on a publicly visible website;
a target patient identification subsystem to identify a target geographic region for targeted advertisement based on a weighted function of targeting factors for each of a plurality of geographic regions within a travel threshold of a medical facility of the practitioner, wherein the targeting factors comprise:
(i) profitability metrics of prior patients,
(ii) average household income metrics,
(iii) an estimated total market share metric of a type of procedure offered by the practitioner, and
(iv) an estimated percentage metric of the total market share already captured by the practitioner;
an advertisement management subsystem to authorize electronic distribution of an advertisement within the target geographic region;
a tracking subsystem to identify a conversion rate of viewers of the advertisement that become new patients;
a profitability return on advertising spend (PROAS) calculation subsystem to calculate a profitability metric of the new patients acquired via the advertisement in the target geographic region; and
a profitability subsystem to adjust weightings applied to the targeting factors based on the calculated profitability metric.

2. The system of claim 1, wherein the profitability subsystem utilizes a machine learning model trained using associations between the weightings applied to the targeting factors and the profitability metric calculated by the PROAS calculation subsystem to adjust the weightings applied to the targeting factors.

3. The system of claim 1, further comprising a reporting subsystem to generate a graphical user interface (GUI) to illustrate profitability metrics and residence location of current patients overlaid on a map.

4. The system of claim 1, further comprising a reporting subsystem to generate a graphical user interface (GUI) to illustrate the metrics associated with the targeting factors of each of the plurality of geographic regions overlaid on a map.

5. The system of claim 1, wherein the advertisement management subsystem is further configured to verify that the advertisement does not conflict with an advertisement of a different practitioner utilizing the patient acquisition system.

6. The system of claim 1, wherein the advertisement management subsystem is further configured to verify that the practitioner has available budget to pay for the electronic distribution of the advertisement within the target geographic region.

7. The system of claim 1, wherein the practitioner reputation management subsystem directs existing patients, subsequent to the procedure performed by the practitioner, to post reviews on at least one of a plurality of publicly visible websites.

8. The system of claim 7, wherein the practitioner reputation management subsystem directs a first set of existing patients to post a review on a first publicly visible website and directs a second set of existing patients to post a review on a second publicly visible website.

9. The system of claim 1, further comprises a practitioner interface subsystem to generate a graphical user interface (GUI) to enable a practitioner to adjust a marketing spend budget and to visualize the calculated profitability metric of the new patients acquired via the advertisement in the target geographic region.

10. The system of claim 1, further comprising a practice management interface subsystem to interface with an existing practice management system of the practitioner.

11. A non-transitory computer-readable medium with instructions stored thereon that, when executed by a processor of a computing system, cause the computing system to implement operations for targeted patient acquisition, the operations comprising:
requesting that existing patients of a practitioner post a review on a publicly visible website following a procedure performed by the practitioner on each respective patient;
identifying a target geographic region for targeted advertisement based on a weighted function of targeting factors for each of a plurality of geographic regions within a travel threshold of a medical facility of the practitioner, wherein the targeting factors comprise:
(i) average household income metrics, and
(ii) an estimated percentage metric of a total market share currently captured by the practitioner;
authorizing electronic distribution of an advertisement within the target geographic region;
identifying a conversion rate of viewers of the advertisement that become new patients;
calculating a profitability metric of the new patients acquired via the advertisement in the target geographic region; and
adjusting weightings applied to the targeting factors based on the calculated profitability metric.

12. The non-transitory computer-readable medium of claim 11, wherein the targeting factors further comprise profitability metrics of prior patients.

13. The non-transitory computer-readable medium of claim 11, wherein the targeting factors further comprise average demographic metrics of individuals within each of the plurality of geographic regions within the travel threshold.

14. The non-transitory computer-readable medium of claim 13, wherein the demographic metrics of individuals comprise one or more of: age, gender, marital status, number of children, race, occupation, and education level.

15. A computer-implemented method for targeted acquisition of clients, comprising:
detecting that a client has received professional services from a professional service provider;
requesting that the client provide a review for posting on a website containing reviews of many professional service provides;
identifying a target geographic region for targeted advertisement based on a weighted function of targeting factors for each of a plurality of geographic regions within a travel threshold of an office of the professional service provider;
authorizing distribution of an advertisement within the target geographic region;
calculating a profitability metric of new clients acquired via the advertisement in the target geographic region; and
adjusting the weightings applied to the targeting factors based on the calculated profitability metric.

16. The method of claim 15, wherein the targeting factors comprise:
profitability metrics of prior clients that live in each of the plurality of geographic regions;
average household income metrics for households in each of the plurality of geographic regions;
estimated total market share metrics of services offered by the professional service provider in each of the plurality of geographic regions; and
estimated percentage metrics of the total market share of the services already captured by the professional service provider in each of the plurality of geographic regions.

17. The method of claim 16, wherein the targeting factors further comprise demographic metrics for each of the plurality of geographic regions.

18. The method of claim 17, wherein the demographic metrics comprise one or more of: age, gender, marital status, number of children, race, occupation, and education level.

19. The method of claim 15, further comprising:
training a machine learning model with associations between weightings applied to the targeting factors and calculated profitability metrics of the clients acquired via the advertisement distributed within the target geographic region.

20. The method of claim 15, further comprising:
distinguishing between positive and negative reviews provided by the clients requested to provide reviews; and
posting only the positive reviews to the website containing reviews.
